(19) United States
(12) Patent Application Publication
(10) Pub. No.: US 2003/0004253 A1
(43) Pub. Date: Jan. 2, 2003

(54) Extraction of hydrocarbons and articles made therefrom

(76) Inventor: John Y. Chen, Pacifica, CA (US)

Correspondence Address: Applied Elastomerics, Inc., 163 W. Harris Avenue, South San Francisco, CA, 94080, US

(21) Appl. No.: 09/896,047

(22) Filed: Jun. 30, 2001

(51) Int. Cl. C08K 5/01; C07C 7/10; C08K 5/24

(52) U.S. Cl. 524/474; 585/833; 524/261

(57) ABSTRACT

A clean and simple process of extracting hydrocarbons from oil containing materials requiring little or no energy.

### FIELD OF THE INVENTION

The present invention relates to extraction of hydrocarbons and the manufacture of novel useful compositions and articles.

### BACKGROUND OF THE INVENTION

Conventional methods of extracting selected organic from mixtures are energy intensive. Therefore, there is a great need to find methods of extracting liquid hydrocarbons requiring little or no energy. The closest known prior art is Burger, et al., U.S. Pat. No. 5,630,474 for a process of extracting crude oil.

### SUMMARY OF THE INVENTION

I have discovered an unusual effect which can be utilized for the extraction of hydrocarbons from (1) liquid-liquid organic mixtures, (2) liquid solid organic mixtures, (3) liquid solid organic/inorganic mixtures, (4) hydrocarbon-polymer gel mixtures, and (5) when said (1)-(4) mixtures are in a vapor-liquid, a vapor-solid, or a vapor-liquid-gel state; said hydrocarbon extractions of mixtures (1) through (5) can be performed using very little or no energy. The method of extraction involves differences in density, differences in surface and interfacial tensions, cohesive forces two or more liquids, and adhesive forces of liquid-solid surfaces under gravitational, buoyancy, or under other physical forces including acceleration and angular forces. Gel articles and gel composite articles with selected amount of oil extracted from its exterior surfaces can provide a higher rigidity gel exterior region or skin while maintain a lower rigidity interior.

The various aspects and advantages of the invention will become apparent to those skilled in the art upon consideration of the accompanying disclosure.

### DESCRIPTION OF THE DRAWINGS

FIG. 1. Representative components materials of composites forming useful articles of the i nvention.

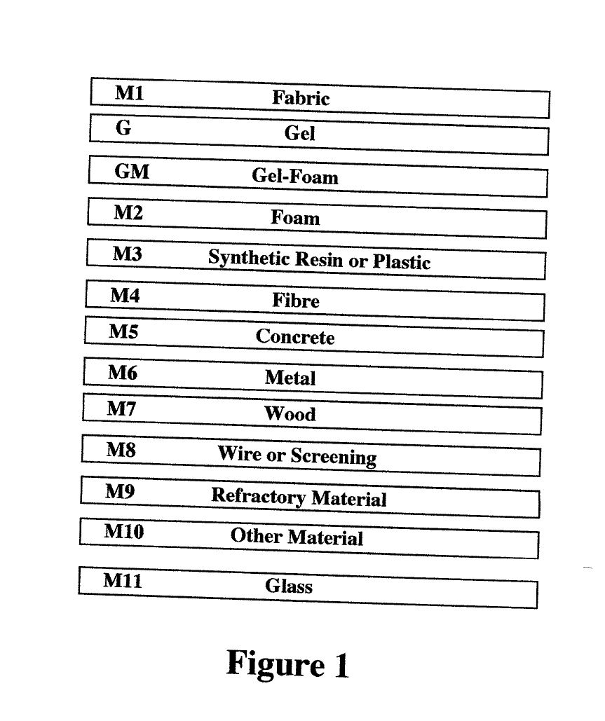

FIG. 2. Representative sectional view of composite articles of the invention (FIG. 2a.=MGM, FIG. 2b.=GMG, FIG. 2c.=MGMGMGM, FIG. 2d.=foam entirely interlocked with composition).

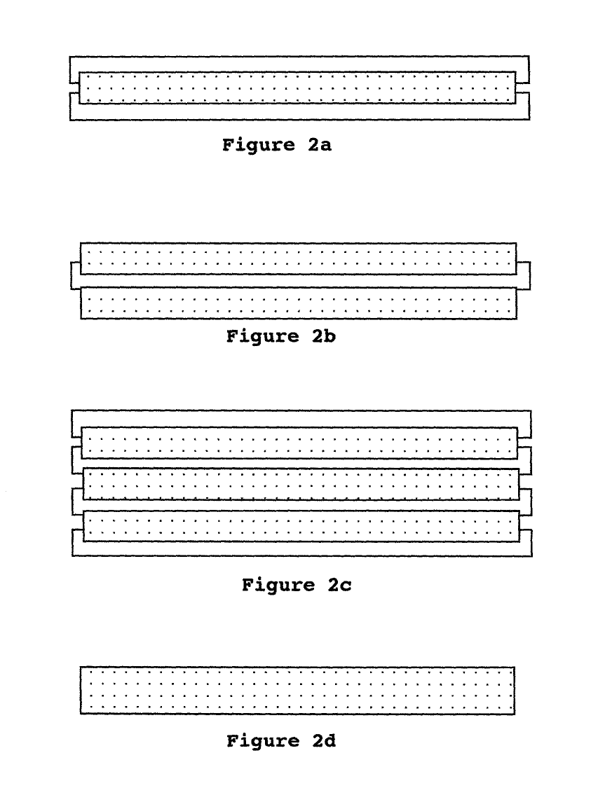

FIGS. 3a-3m. Representative sectional view of composite articles as shown generally by the relationship of Gn and Mn and more specific article examples of M1, M2, M3, and M4 with Gn when the material Mn is n=1 (fabric/cloth), n=2 (foam/sponge), n=3 (synthetic resin/plastic), and n=4 (fibre) as shown in FIGS. 3d, 3e, 3h, and 3j respectively.

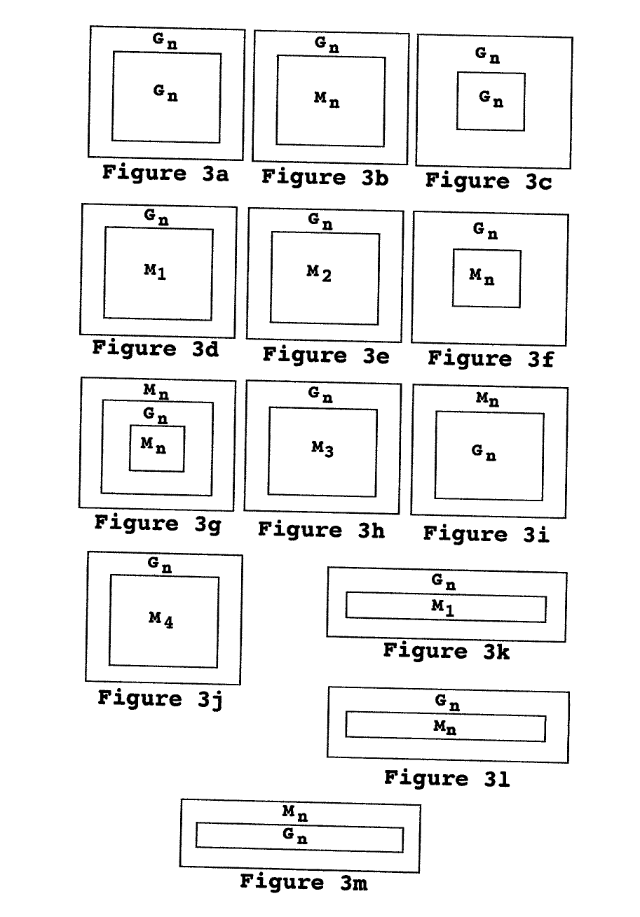

FIGS. 4a-4n. Representative sectional view of composite articles as shown generally by the relationship of Gn and Mn and more specific article examples of M1, M2, M3, and M4 with Gn when the material Mn is n=1 (fabric/cloth), n=2 (foam/sponge), n=3 (synthetic resin/plastic), and n=4 (fibre) as shown in FIGS. 4l, 4m, and 4n respectively.

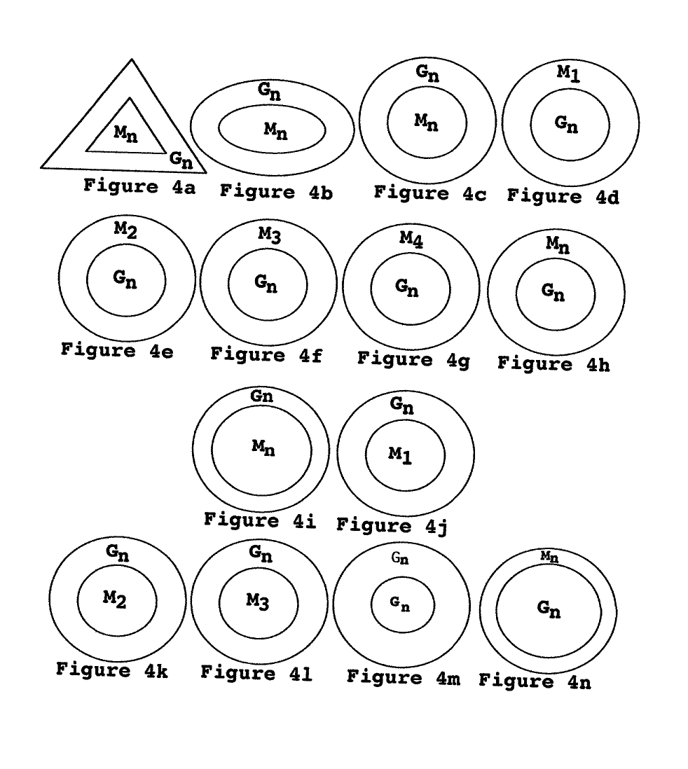

### DESCRIPTION OF THE INVENTION

I have discover that the liquid hydrocarbons and certain additives contained in highly extended thermoplastic elastomer gels plasticized with oils can be extracted, separated, or removed from such gels by the use of a semi porous membrane utilizing little or no energy. The most suitable semi porous membrane is found to be a liquid coating of silicone fluid, other suitable membrane materials include silicone greases, silicone gels, silicone elastomers, and their combinations which are herein described below.

With respect to thermoplastic elastomer gels, some are described in my U.S. Pat. Nos. 6,161,555, 6,148,830, 6,117,176, 6,050,871, 6,033,283, 5,962,572, 5,938,499, 5,884,639, 5,868,597, 5,760,117, 5,655,947, 5,633,286, 5,624,294, 5,508,334, 5,475,890, 5,336,708, 5,334,646, 5,324,222, 5,239,723, 5,153,254, 5,262,468, 4,618,213, and 4,369,284 including references cited in the above patents. Other oil gel and oil-polymer composition patents include U.S. Pat. Nos. 2,830,402, 4,136,699, 3,485,787, 3,676,387, 3,827,999, 4,151,057, 4,176,240, 4,259,540, 4,492,428, 4,864,677, 4,880,878, 4,905,337, 4,920,662, 4,975,999, 5,994,446, 5,952,396, 5,929,138, 5,879,694, 5,863,977, 5,849,824, 5,559,165, 5,459,193, 5,442,004, 5,360,350, 4,716,183, 4,709,982, 5,710,206, 5,618,882, 5,541,250, 5,626,657, 5,830,237, 5,888,216, 5,925,707, and 5,994,450. The above cited patents are incorporated herein by reference.

Polymers plasticized with oil are many including SIS, SBS, SEBS, SEP, SI, SB, SEPSEP, high vinyl SEBS, (SEB)2-X-(I)2, (SB)2-X-(B)2, star diblock SEPS, SEEPS, SEB/EPS, SIBS, ESE, Dow E/S interpolymers, EPDM terpolymers (Nordel IP made from INSITE constrained geometry catalyst), and the like.

Oil plasticizers forming gels of the above cited polymers include rubber processing oils such as paraffinic and naphthenic petroleum oils, highly refined aromatic-free paraffinic and naphthenic food and technical grade white petroleum mineral oils, and synthetic liquid oligomers of polybutene, polypropene, polyterpene, etc. The synthetic series process oils are high viscosity oligomers which are permanently fluid liquid nonolefins, isoparaffins or paraffins of low, moderate to high molecular weights.

The amount of plasticizing oils contained in the gels can vary depending on the gel rigidity of the gel which rigidity can range from less than 1 gram Bloom to about 3,000 gram Bloom and higher. As used herein, the term “gel rigidity” in gram Bloom is determined by the gram weight required to depress a gel a distance of 4 mm with a piston having a cross-sectional area of 1 square centimeter at 23° C.

The amount of plasticizer incorporated into the polymer gels (based on 100 parts by weight of polymer) cited above can range from about 1.0 part by weight to about 3,000 parts by weight and higher including about 2, 5, 10, 20, 30, 40, 50, 100, 200, 300, 400, 500, 600, 700, 800, 900, 1,000, 1,500, 2,000 and the amount of plasticizer may also range in values between such values of parts by weights.

Examples of representative commercially available plasticizing oils include Amoco® polybutenes, hydrogenated polybutenes, polybutenes with epoxide functionality at one end of the polybutene polymer, liquid poly(ethylene/butylene), liquid hetero-telechelic polymers of poly(ethylene/butylene/styrene) with epoxidized polyisoprene and poly(ethylene/butylene) with epoxidized polyisoprene: Example of such polybutenes include: L-14 (320 Mn), L-50 (420 Mn), L-100 (460 Mn), H-15 (560 Mn), H-25 (610 Mn), H-35 (660 Mn), H-50 (750 Mn), H-100 (920 Mn), H-300 (1290 Mn), L-14E (27-37 cst@100° F. Viscosity), H-300E (635-690 cst@210° F. Viscosity), Actipol E6 (365 Mn), E16 (973 Mn), E23 (1433 Mn), Kraton L-1203, EKP-206, EKP-207, HPVM-2203 and the like. Example of various commercially oils include: Duraprime and Tufflo oils (6006, 6016, 6016M, 6026, 6036, 6056, 6206, etc), other white mineral oils include: Bayol, Bernol, American, Blandol, Drakeol, Ervol, Gloria, Kaydol, Litetek, Lyondell (Duraprime 55,70, 90, 200, 350, 400), Witco white oils including 40 oil, Marcol, Parol, Peneteck, Primol, Protol, Sontex, Petrolia oil, hydrobrite 200 PO, 380 PO, 550 PO, and the like.

Generally, plasticizing oils with average molecular weights less than about 50 and to greater than about 2,000 may also be used (e.g. H-300 (1290 Mn)). It is well know that minor and sufficient amounts of Vitamin E is added to the described commercially available oils during bulk processing which is useful as a oil stabilizer, antioxidant, and preservative.

Of all the factors, for example is a gel floss, the amount of plasticizing oils can be controlled and adjusted advantageously to obtain substantially higher tear and tensile strength gels. The improvements in tensile strength of the gels are accompanied by corresponding increase in gel rigidity as the amount of plasticizing oils are lowered until the rigidity of the gels becomes much higher than that of the gums which surround the teeth. Although higher tensile strengths can be obtained as the amount of plasticizing oils in the gel approaches zero, the tensile strength of the floss, however, must be maintained at an acceptable gel rigidity (at sufficient high plasticizing oil levels) in order to be as soft as the gums required for flossing. For example, the rigidities of a gel containing 100, 200, or 300 parts by weight of oil is much higher than a gel containing 300, 400, 500, 600, 800, or 900 parts of oil.

These gels can exhibit a larger unit lateral contraction at the same elongation per unit of length as their counterpart parent gels from which the new gels are derived or formed. This property would allow a same unit volume of gel when elongated as its parent to easily wedge between the teeth when flossing. It would seem that a gel having the 1.0 cm³ volume made from a ratio of 100 parts by weight of copolymer and 400 parts plasticizer would have a unique macro volume configurations that is at equilibrium with the plasticizer which is much like a 3-D fingerprint which is uniquely different from any other gel of a different copolymer to plasticizer ratio. Reducing the plasticizer content of a ratio 100:400 gel to a 100:300 ratio of copolymer to plasticizer will decrease the amount of plasticizer, but the original macro volume configurations will remain the same.

Speculative theories not withstanding, configurations may take the form of (1) swiss cheese, (2) sponge, (3) the insides of a loaf of bread, (4) structures liken to ocean brain corals, (5) large structures and small structures forming the 3-D gel volume landscape, (6) the outer heated surface which cools faster than the inner volumes of the gel during its cooling histories may have a patterned crust (rich in A micro-phases) like that of a loaf of bread and the inner volume may have much like 1-5, and (7) the many different possible structures are unlimited and volume landscapes may be interconnected at the macro-level by threads or micro-strands of Z micro-phases.

The ability to make gels with a continuous gradual varying rigidity index without any physical discontinuity boundary is advantageous. For example, it would be desirable to form a gel ball with a higher rigidity on the outer surface to increase wear resistance without first forming a low rigidity gel center and dipping or coating the low rigidity gel ball center with a higher rigidity gel outer layer. In any event, the coating of a higher rigidity outer gel layer onto a lower rigidity softer ball core require additional steps and energy and the outer higher rigidity gel coat can peel off like an outer layer of an onion with a physical discontinuity or physical boundary at which line the two separate gel regions can be peeled apart. The center gel core does not form an integral continuous, boundary less composition with the outer higher rigidity layer no matter how high the bath temperature of higher rigidity gel is heated prior to dipping or coating onto the center gel core. The outer core will always peel off when peeling force is applied at the interface between the outer layer and the gel core. There is no way around this problem. This is also the case for a ball coated with the same rigidity gel. There is no difference if the gel center core is coated on the outside with a lower rigidity, higher rigidity, or even the same rigidity gel, the outer coating will always peel off the ball when a sufficient peeling force is applied at the interface. This is also the case for any gel article of any shape coated with one or more layer of another gel including strands for flossing. The outer layer will always peel off when sufficient peeling force is applied at the gel-gel interface.

Therefore it is of great advantage to be able to extract oil from the outer volume or areas of a gel article to improve to increase the volume or area rigidity and its outer surface wear properties and at the same time not creating any internal or external gel-gel peelable interface. It is also of great advantage to be able to reduce, reduce substantially, or extract substantially all the oil from the entire volume of the gel article to reduce or remove the oil volume of any gel article without creating any internal or external gel peelable interfaces thereby creating an entirely new gel article of reduced oil volume or content and article of greater rigidity.

In my U.S. Pat. No. 6,148,830, at col. 12, lines 28-54, I describe the advantages of extracting plasticizer from gels. The methods of plasticizer extraction from already formed gels all require the input of additional energy. For example, (1) soxhlet extraction involving solvent exchange require additional electrical and heat energy, (2) pressure-heat extraction require additional electrical and heat energy, (3) vacuum-heat-pressure extraction require additional electrical and heat energy, and (4) vacuum-solvent and vacuum-heat-solvent-pressure extractions also require additional electrical and heat energy.

Therefore, it is advantageous to perform extractions of oil from oil gels using very little or no additional energy.

The invention is also suitable for extracting to all types of oils and hydrocarbons in any form, such as from extremely high, and high viscosity liquid-liquid hydrocarbon mixtures or liquid-solid hydrocarbon mixtures (for example: crude and other fossil containing liquid hydrocarbons such as tar) utilizing little or no energy is of great advantage. The extraction or separation of selected hydrocarbons from raw crude oil or other hydrocarbon mixtures obtained from under the earth's surface and performing such extraction to obtain useful hydrocarbon values with little or no energy can realized by the method of the present invention.

The extraction, separation, and removal of hydrocarbons, especially hydrocarbon oils from oil extended polymer gels and the extraction, separation, and removal of useful hydrocarbon fractions (oils) from high viscosity liquid-liquid and liquid-solid crude mixtures can be obtained by the use of one or more of a liquid, liquid-solid, liquid-solid supported, or solid silicone membrane. The useful membrane can be in the form of (1) one or more silicone fluids of low, moderate, high, or extremely high viscosity, (2) one or more silicone fluids in combination with one or more of a porous substrate, (3) one or more silicone fluids in combination with one or more of a solid silicone membrane, and (4) one or more silicone fluids in combination with one or more of a porous substrate and silicone solid membrane.

Silicones are semi organic, synthetic polymers comprising alternating silicon and oxygen or polyorganosiloxanes in the form of fluids, elastomers, gels, and resins. Silicone elastomers are based on dimethylsiloxane polymers made by the polymerization of octamethylcyclotetrasiloxane. Copolymers are added to enhance properties: methylvinylsiloxane groups improves vulcanization and compression-set resistance; phenyl groups improve low-temperature properties; and trifluoropropyl groups provide fuel and solvent resistance. Other silicon based block and copolymers include those described in U.S. Pat. Nos. 6,225,390, 46,174,968 and 56,160,045. Uncured silicone elastomers in the form of paste, grease, gums and their cured form are all suitable for use in the present invention include GE Silicones: HCE, Silipren LSR silicones, LIM 8040,9070, LSR 20X0, 2XX0, 21X0, 22XX, 2345/0Y′, 25X0, 26X0, 60XX, 26X1, 27X0, 29X0, 3Z85/XX, 3Z86/XX, 40X0, 5Y50, FSL 7208, 7210, Electro 242-V, Electro 245, Electro 2X0, HCR, Silplus GE silicones Silplus SE6035, 6075, 6740, 6160, 6180, 6740, 6750 ,6770, 6335, 6350, 6370, 6260, etc.

Commercially available silicone fluids, gels and greases based on dimethylsilicone or polydimethylsiloxane (clear low to high viscosity liquids) include Dow Corning 200 fluid food grade, 200 fluid high viscosity, 203 fluid, 230 fluid, MB50-001, MB50-002, MB50-004, MB50-010, MB50-011, BY27-006, BY27-007, 4-7105, 4-7051, 4-7081, 1-9641, silicone oil 42,000; GE Silicones G623, G624, G661, Specialty Product KantSilk 406 NOD, KantSilk M-55, Witco, CP Hall L-42 and L-45, GE Silicones Harwick Standard SF96, SF 1080, Viscasil, and SF 18-350; TSF451-100. The viscosity of such silicone fluids range from less than about less than 1.0 cSt, to about 100,000 cSt and higher including viscosity values (centistoke) of 1.0 cSt., 2.0 cSt, 5.0 cSt, 10 cSt., 20 cSt, 50 cSt, 100 cSt, 200 cSt, 300 cSt, 350 cSt, 400 cSt, 500 cSt, 600 cSt, 700 cSt, 800 cSt, 900 cSt, 1,000 cSt, 2,000 cSt, 3000 cSt, 4000 cSt, 5000, cSt, 6000 cSt, 7000 cSt, 8000 cSt, 9000 cSt, 10,000 cSt, 20,000 cSt, 30,000 cSt, 40,000 cSt, 50,000 cSt, 60,000 cSt, 70,000 cSt, 80,000 cSt, 90,000 cSt, and 100,000 cSt, 200,000 cSt, 300,000 cSt and higher; PolySi Tech., Inc. silicone greases: PST-503, 504, 507, 511, 515, 516, 524, 535, 540, 552, 555, 587, 597, 599, PST-444, 433, 461, 455; fluids PST-801, 803, 805, 810, 811, 813, 815, 816, 822, 828, 831, 841, 846, 851, 50; NuSil: silicone gels MED10-6300, 12-6300, 6340; silicone fluids MED-360,420, CV-7300, greases CV-9042, 9342, silicone potting gel CV-8151, 1-8151, 8251; Rhodia: Rhodorsil polymer A, fluid 621V1000, 621V(3,500), 621V200, 48V100, 48V50, Hydrofugent 68, 621V600, 47V500, 47V5, 47V(3,000), 48V3500, 47V100, 47V10, 47V200, 47V20, 47V(1,000), etc. Blends of such silicone fluids, gels, and greases can produce almost any intermediate viscosity values, surface tension values, and density as desired.

The present invention should not be held to any speculative theory. From the experiments and current understanding of viscosity, intermolecular forces of cohesion and adhesion, surface and interfacial tension, density, gravity, and buoyancy, a theory can be made to explain the physics involved in the extraction process which reasoning is as follows:

(1) When water is placed in contact with an oil extended gel, the gel will not over time exhibit weight loss.

(2) When oil is add to a column of water in a test tube, the oil will separate out and find its level above the column of water.

(3) The surface tension of water at 25° C. is about 72.0 mN/m.

(4) The surface tension of oil (mineral oil) at 25° C. is about 29.7 mN/m.

(5) The surface tension of silicone fluid at 25° C. range from abut 16 to abut 22 mN/m (for example: the surface tension of 100 cSt silicone fluid at STP is 20.9 mN/m).

(6) The density of oil is less than the density of silicone fluid, silicone grease, silicone gel, and silicone elastomer.

(7) Oil is not a polar liquid and is highly compatible with the rubber phase of the oil gel forming polymer.

(8) Silicone is polar and not compatible with the polymer's rubber phase.

The molecules of a liquid oil drop attract each other. The interactions of an oil molecule in the liquid oil drop are balanced by an equal attractive force in all directions. Oil molecules on the surface of the liquid oil drop experience an imbalance of forces at the interface with air. The effect is the presence of free energy at the surface. This excess energy is called surface free energy and is quantified as a measurement of energy/area. This can be described as tension or surface tension which is quantified as a force/length measurement or m/Nm.

Clearly gravity is the only force pulling on the extracted oil from the gel in the presence of silicone fluid at the gel-petri dish interface in the examples below. In the case of gel samples in the petri dishes in contact with silicone fluids, the extracted oil are collected on the top surface layer of the silicone fluid while the silicone fluid maintain constant contact and surrounds the gel sample. In the case of gel placed in a test tube of silicone fluid of different viscosity, the oil is extracted and migrates and collect at the top of the silicone fluid surface while the gel reduces in volume with time. The oil extraction process in silicone is accompanied by buoyant forces removing the extracted oil from the surroundings of the gel constantly surrounding the gel with fresh silicone fluid while in the example of alcohol, since the oil is heavier, the oil is maintained and surrounds the gel sample forming a equilibrium condition of oil surround the gel sample while keeping the alcohol from being in contact with the gel sample. Therefore in order to use alcohol to extract oil from a gel sample, the extracted oil must be constantly removed from the oil alcohol mixture as is the case during soxhlet extraction which process requires additional energy to pump the oil-alcohol mixture away from the sample and removing the oil before forcing the alcohol back to the gel sample surface to perform further extraction.

Silicone fluid is efficient and useful for extracting oil form oil gel compositions with the assistance of gravity and buoyancy of oil in the silicone fluids.

It is very difficult to extract, separate, or remove oil from an oil gel composition by positive or vacuum pressure or heat while using little or no energy and because of the affinity of the rubber midblock for oil, not even the weight of a two ton truck resting on a four square foot area (placing a layer of gel between four pairs of one foot square parallel steel plates one set under each of the truck tire resting on the gels) can separate the oil from the gel composition.

The use of silicone fluids of various viscosity acts as a liquid semi porous membrane when placed in constant contact with an oil gel composition will induce oil to migrate out of the gel composition. By the use of gravity or oil buoyancy, no energy is required run the oil extraction process.

In the case of an oil extended polymer, say in the shape of an oil gel ball as describe in SEEPS Example 16, the rubber being highly compatible with the oil, holds the oil in place within the boundary of the rubber molecular phase. It is this affinity of the (1) rubber and oil molecules and (2) the attraction of oil molecules for each other that prevents the oil from bleeding out of the surface of the oil extended polymer ball. There exist then, at the surface of the oil extended polymer ball several types of surface tensions of: (1) oil-air surface tension, (2) oil-rubber surface tension, (3) rubber-air surface tension, (4) rubber/oil-air surface tension, and (5) rubber-rubber surface tension. Other forces acting on the oil extended polymer ball are: the elastic force of the polymer network pulling inwards, similar to stretched out rubber bands, which is in equilibrium with the oil molecules' attraction to the rubber molecules of the polymer network. In the case of SBS, the lower compatibility of the midblock butadiene with oil, once the ball is made, the SBS network immediately contracts due to elastic forces to produce oil bleeding which is evidence of the poor compatibility of the rubber block for the oil molecules.

The intermolecular forces that bind similar molecules together are called cohesive forces. Intermolecular forces that bind a substance to a surface are called adhesive forces.

When two liquids are in contact such as oil and silicone fluid, there is interfacial tension. The more dense fluid is referred to herein as the “heavy phase” and the less dense fluid is referred to as the “light phase”. The action at the surface of the oil extended polymer gel surface when brought into contact with silicone fluid is as follows: a drop of silicone fluid when placed on the flat surface of a oil extended polymer gel will wet the gel surface and spread over a larger area as compared to a drop of oil placed on the same gel surface. Because the surface free energy of the silicone fluid in contact with the gel surface is lower than the surface free energy of the oil, the silicone fluid has the ability to displaces the oil from the surface of the gel.

A thermoplastic oil gel composite article comprising a gel with at two or more rigidity regions can be made by the method of the invention, said gel rigidity regions having no physically separable boundaries, wherein said gel is being denoted by G, is physically interlocked with a selected material M or in combination with one or more of a different gel forming a composite of the combination GnGn, GnGnGn, GnMn, GnMnGn, MnGnMn, MnGnGn, MnMnMnGnMn, MnGnGnMn, GnMnGnGn, GnGnMnMn, GnMnMnGn, GnGnMnGnMnGnGn, GnMnGnMnMn, MnGnMnGnMnGn, GnGnMnMnGn, GnGnMnGnMn, GnGnMnGnMnGn, GnMnGnMnGn, MnMnMnGn or a permutation of one or more of said Gn with Mn; wherein when n is a subscript of M, n is the same or different selected from the group consisting of paper, foam, plastic, fabric, metal, metal foil, concrete, wood, glass, glass fibers, ceramics, synthetic resin, synthetic fibers or refractory materials; and wherein when n is a subscript of G, n denotes a different gel rigidity.

### Buoyancy can be use to Advantage

Further, because the silicone fluid phase is heavier than the oil phase, the lighter oil phase is remove from the surface of the gel because of buoyancy and is transported away and above the heavier silicone fluid line level.

### Gravity can be use to Advantage

A highly viscous silicone fluid can be use to advantage. When the bulk amount of the high viscosity silicone fluid is too great to allow the oil molecules to migrate across then, the removal and transport of the oil away from the surface of the gel is slowed so as to form an equilibrium of oil-silicone fluid at the gel surface. A thin coat of a high viscosity silicone fluid appears to work best to provide for removal and transport of the oil away from the surface of the oil extended polymer gel surface by gravity. Such a lightly coated polymer gel surface will drip oil when held vertically above a collecting container or when placed on an inclined position, oil will run downward from the gel-container surface interface.

The present invention is useful for making gel articles which are designed to have a soft, lower rigidity interior and a tough or higher rigidity thick exterior volume or a higher rigidity thin exterior skin. The transformation can be achieved with the use of little or no additional electrical or heat energy. Such products include: toys; games; novelty, or souvenir items; elastomeric lenses, light conducing articles, optical fiber connectors; athletic and sports equipment and articles; medical equipment and articles including derma use and for the examination of or use in normal or natural body orifices, health care articles; artist materials and models, special effects; articles designed for individual personal care, including occupational therapy, psychiatric, orthopedic, podiatric, prosthetic, orthodontic and dental care; apparel or other items for wear by and on individuals including insulating gels of the cold weather wear such as boots, face mask, gloves, full body wear, and the like have as an essential, direct contact with the skin of the body capable of substantially preventing, controlling or selectively facilitating the production of moisture from selected parts of the skin of the body such as the forehead, neck, foot, underarm, etc; cushions, bedding, pillows, paddings and bandages for comfort or to prevent personal injury to persons or animals; housewares and luggage; articles useful in telecommunication, utility, industrial and food processing, and the like.

The teachings of the invention can also apply to the extraction of selected hydrocarbons from (1) liquid-liquid organic mixtures, (2) liquid solid organic mixtures, (3) liquid solid organic/inorganic mixtures, (4) hydrocarbon-polymer gel mixtures, and (5) when said (1)-(4) mixtures are in a vapor-liquid, a vapor-solid, or a vapor-liquid-gel state; said hydrocarbon extractions of mixtures (1) through (5) can be performed using very little or no energy. Such extractions include extraction of petroleum crude oil including heavy crude oil and medium crude oil feedstock, extraction of oil from oil spill clean up materials, extraction of oil values from food seeds, beans, and grains, extraction of oil from biomass, extraction of oil from oil mist collectors, extraction of oil from shale oil, and the like.

While advantageous components and formulation ranges based on the desired properties of the multiblock copolymer gels have been disclosed herein. Persons of skill in the art can extend these ranges using appropriate material according to the principles discussed herein. All such variations and deviations which rely on the teachings through which the present invention has advanced the art are considered to be within the spirit and scope of the present invention.

The invention is further illustrated by means of the following illustrative embodiments, which are given for purpose of illustration only and are not meant to limit the invention to the particular components and amounts disclosed.

### EXAMPLE 1

When 4.1 gram of a oil extended gel made from 600 parts by weight of Witco 40 oil and 100 parts by weight of Septon SEEPS 4055 is placed in a glass test tube containing Dow coming dimethylpolysiloxane 200 fluid having a 12,500 cSt viscosity and heated to about 300° F. for 30 minutes, the resulting measured gel weight was found to be 3.1 grams resulting in a weight loss of 1.0 gram of oil from the gel. The extracted oil from the gel sample was observed suspended above the silicone fluid level in the test tube after the gel sample was removed.

### EXAMPLE 2

When 4.1 gram of a oil extended gel made from 600 parts by weight of Witco 40 oil and 100 parts by weight of Septon SEEPS 4055 is placed in a glass test tube containing Dow coming dimethylpolysiloxane 200 fluid having a 350 cSt viscosity and heated to about 300° F. for 30 minutes, the resulting measured gel weight was found to be 2.51 grams resulting in a weight loss of 1.59 gram of oil from the gel. The extracted oil from the gel sample was observed suspended above the silicone fluid level in the test tube after the gel sample was removed.

### EXAMPLE 3

When 3.8 gram of a oil extended gel made from 800 parts by weight of Duraprime 90 and 100 parts by weight of Kraton SEBS 1651 is placed in a glass test tube containing Dow coming dimethylpolysiloxane 200 fluid having a 20 cSt viscosity and heated to about 300° F. for 30 minutes, the resulting measured gel weight was found to be 1.7 grams resulting in a weight loss of 2.1 gram of oil from the gel. The extracted oil from the gel sample was observed separated from the silicone fluid level in the test tube after the gel sample was removed by slowly stirring the clear liquid column producing clear turbulence at the separation level, otherwise no observable separation line was apparent to the eye between the oil and silicone fluid when the column was at rest.

### EXAMPLE 4

When 2.9 gram of a oil extended gel made from 800 parts by weight of Duraprime 90 and 100 parts by weight of Kraton SEBS 1651 is placed in a glass test tube containing Dow coming dimethylpolysiloxane 200 fluid having a 50 cSt viscosity and heated to about 300° F. for 30 minutes, the resulting measured gel weight was found to be 1.93 grams resulting in a weight loss of 0.97 gram of oil from the gel. The extracted oil from the gel sample was observed suspended above the silicone fluid level in the test tube after the gel sample was removed. The oil appear above the clear silicone fluid line due to difference in index of refraction's.

### EXAMPLE 5

When 3.1 gram of a oil extended gel made from 800 parts by weight of Duraprime 90 and 100 parts by weight of Kraton SEBS 1651 is placed in a glass test tube containing Dow coming dimethylpolysiloxane 200 fluid having a 100 cSt viscosity and heated to about 300° F. for 30 minutes, the resulting measured gel weight was found to be 1.58 grams resulting in a weight loss of 1.52 gram of oil from the gel. The extracted oil from the gel sample was observed suspended above the silicone fluid level in the test tube after the gel sample was removed. The oil appear above the clear silicone fluid line due to difference in index of refraction's.

### EXAMPLE 6

When 3.35 gram of a oil extended gel made from 800 parts by weight of Duraprime 90 and 100 parts by weight of Kraton SEBS 1651 is placed in a glass test tube containing Dow coming dimethylpolysiloxane 200 fluid having a 200 cSt viscosity and heated to about 300° F. for 30 minutes, the resulting measured gel weight was found to be 1.92 grams resulting in a weight loss of 1.43 gram of oil from the gel. The extracted oil from the gel sample was observed suspended above the silicone fluid level in the test tube after the gel sample was removed. The oil appear above the clear silicone fluid line due to difference in index of refraction's.

### EXAMPLE 7

When 2.8 gram of a oil extended gel made from 800 parts by weight of Duraprime 90 and 100 parts by weight of Kraton SEBS 1651 is placed in a glass test tube containing Dow coming dimethylpolysiloxane 200 fluid having a 350 cSt viscosity and heated to about 300° F. for 30 minutes, the resulting measured gel weight was found to be 2.15 grams resulting in a weight loss of 0.65 gram of oil from the gel. The extracted oil from the gel sample was observed suspended above the silicone fluid level in the test tube after the gel sample was removed. The oil appear above the clear silicone fluid line due to difference in index of refractions. This experiment is repeated by placing a weighted gel sample in a test tube under at least 2.5 cm head of 350 cSt fluid at room temperature for 30 hours, the gel exhibited no weight loss. Whereas when a weight gel sample is dipped and coated with 350 cSt fluid and then placed in a petri dish with about ½ of its volume submerged in the fluid, the weight loss is consistent with Example 13 below. In the petri dish experiments described in the examples, the gel sample is first dipped and coated by turning it over and over in the petri dish containing the appropriate silicone fluid before allowing it to rest and placing the petri dish cover over the sample and fluid.

### EXAMPLE 8

When 3.7 gram of a oil extended gel made from 800 parts by weight of Duraprime 90 and 100 parts by weight of Kraton SEBS 1651 is placed in a glass test tube containing 99% isopropyl alcohol anhydrous SEP at room temperature for 18 hours, the resulting measured gel weight was found to be 2.65 grams resulting in a weight loss of 1.05 gram of oil from the gel. The extracted oil from the gel sample was observed suspended below the alcohol fluid level in the test tube after the gel sample was removed. The gel rigidity of the resultant gel appears to be somewhat uniform apparently because the extracted oil surrounds the sample while the alcohol is displaced by the extracted oil to the top portion of the test tube. The extraction of oil from the gel is slowed and frustrated because the extracted oil is not transported away from the vicinity of the gel body and because the oil is denser than alcohol, the oil comes to equilibrium at both the alcohol-oil interface and the free oil-bounded oil gel interface.

### EXAMPLE 9

When 9.4 gram of a oil extended gel made from 600 parts by weight of Witco 40 oil and 100 parts by weight of Septon SEEPS 4055 is placed in a covered #1029 100×15 mm clear petri dish containing Dow coming dimethylpolysiloxane 200 fluid having a 20 cSt viscosity at room temperature for 30 hours, the resulting measured gel weight was found to be 3.1 grams resulting in a weight loss of 6.3 gram of oil from the gel. The extracted oil from the gel sample was observed suspended above the silicone fluid level in the petri dish after the gel sample was removed.

### EXAMPLE 10

When 10.35 gram of a oil extended gel made from 600 parts by weight of Witco 40 oil and 100 parts by weight of Septon SEEPS 4055 is placed in a covered #1029 100×15 mm clear petri dish containing Dow corning dimethylpolysiloxane 200 fluid having a 50 cSt viscosity at room temperature for 30 hours, the resulting measured gel weight was found to be 4.10 grams resulting in a weight loss of 6.25 gram of oil from the gel. The extracted oil from the gel sample was observed suspended above the silicone fluid level in the petri dish after the gel sample was removed.

### EXAMPLE 11

When 9.8 gram of a oil extended gel made from 600 parts by weight of Witco 40 oil and 100 parts by weight of Septon SEEPS 4055 is placed in a covered #1029 100×15 mm clear petri dish containing Dow coming dimethylpolysiloxane 200 fluid having a 100 cSt viscosity at room temperature for 30 hours, the resulting measured gel weight was found to be 3.6 grams resulting in a weight loss of 6.20 gram of oil from the gel. The extracted oil from the gel sample was observed suspended above the silicone fluid level in the petri dish after the gel sample was removed.

### EXAMPLE 12

When 9.1 gram of a oil extended gel made from 600 parts by weight of Witco 40 oil and 100 parts by weight of Septon SEEPS 4055 is placed in a covered #1029 100×15 mm clear petri dish containing Dow corning dimethylpolysiloxane 200 fluid having a 200 cSt viscosity at room temperature for 30 hours, the resulting measured gel weight was found to be 3.7 grams resulting in a weight loss of 5.4 gram of oil from the gel. The extracted oil from the gel sample was observed suspended above the silicone fluid level in the petri dish after the gel sample was removed.

### EXAMPLE 13

When 8.25 gram of a oil extended gel made from 600 parts by weight of Witco 40 oil and 100 parts by weight of Septon SEEPS 4055 is placed in a covered #1029 100×15 mm clear petri dish containing Dow corning dimethylpolysiloxane 200 fluid having a 350 cSt viscosity at room temperature for 30 hours, the resulting measured gel weight was found to be 3.15 grams resulting in a weight loss of 5.1 gram of oil from the gel. The extracted oil from the gel sample was observed suspended above the silicone fluid level in the petri dish after the gel sample was removed.

### EXAMPLE 14

When 10 gram of a oil extended gel made from 600 parts by weight of Witco 40 oil and 100 parts by weight of Septon SEEPS 4055 is placed in a covered #1029 100×15 mm clear petri dish containing witco 40 oil having a 4.3 cSt viscosity Kin., at room temperature for 30 hours, the resulting measured gel weight was found to be 13.10 grams resulting in a weight gain of 3.1 gram of oil from the gel. The extracted oil from the gel sample was observed to be the same as the 40 oil in the petri dish after the gel sample was removed.

### EXAMPLE 15

When 9.7 gram of a oil extended gel made from 600 parts by weight of Witco 40 oil and 100 parts by weight of Septon SEEPS 4055 is placed in a covered #1029 100×15 mm clear petri dish containing Dow corning dimethylpolysiloxane 200 fluid having a 12,500 cSt viscosity at room temperature for 30 hours, the resulting measured gel weight was found to be 8.50 grams resulting in a weight loss of 1.20 gram of oil from the gel. The extracted oil from the gel sample was observed suspended above the silicone fluid level in the petri dish after the gel sample was removed.

### EXAMPLE 16

When 106.65 gram oil extended solid gel ball made from 800 parts by weight of Witco Parol oil and 100 parts by weight of Septon SEEPS 4055 is placed in a cup containing Dow coming dimethylpolysiloxane 200 fluid having a 100 cSt viscosity at room temperature for 12, hours, 24 hours, 30 hours, and 42 hours, the resulting measured gel weight was found to be 95.4, 93.24, 90.70, and 80.91 grams respectively resulting in a weight loss of 11.25, 13.41, 15.95, and 25.74 grams respectively of oil from the gel. The extracted oil from the gel sample was observed suspended above the silicone fluid level in the cup after the gel sample was removed. The rigidity of the outer surface of the solid gel ball increased with oil extraction time in the silicone fluid. When cut open, the higher rigidity was confined on the outer circumference of the solid gel ball.

### EXAMPLE 17

When 6.9 gram of a oil extended gel made from 800 parts by weight of duraprime 90 and 100 parts by weight of Kraton SEBS 1651 is placed in a covered #1029 100×15 mm clear petri dish containing Dow coming dimethylpolysiloxane 200 fluid having a 50 cSt viscosity at room temperature for 6 hours, the resulting measured gel weight was found to be 4.10 grams resulting in a weight loss of 2.8 gram of oil from the gel. The extracted oil from the gel sample was observed suspended above the silicone fluid level in the petri dish after the gel sample was removed.

### EXAMPLE 18

When cube samples of oil extended gel made from Example 15 and 17 are slightly coated on the outside surface with Dow silicone 200 fluid of 350 and 12,500 cSt viscosity respectively and one of each of the two cube's flat surface is placed in separate petri dishes at room temperature and the petri dishes are placed on an incline, the oil contained in the both solid gel samples began immediately to run or drool from the gel-silicone fluid-petri dish resting interface. The extracted oil is observed to continually run down the incline at one of the corner of each of the gel cube resting on the petri dish surface unto the vertical wall of the petri dish container where the oil collects. The interface where the cubes rests on the petri dish's surface appear to be contain a layer of silicone fluid. Observing from the under side of the petri dish, silicone fluid appears to be maintained under the gel cube samples due to capillary action while oil appear to drool from the corner and edge of the bottom surface of the gel cubes.

### EXAMPLES OF GRAVITY EXTRACTION

### EXAMPLE 19

A large porous multi-ply paper hand towel reinforced with large separation netting is saturated, coated, or permeated with 200 cst silicone fluid and a 500 gram oil gel compositions of Examples 15 and 17 are wrapped with the towel tied and hung above a container for collecting the extracted oil due to gravity pulling on the oil extracted by the silicone fluid soaked towel in constant contact with the surface of the oil gel composition.

### EXAMPLE 20

Example 19 is repeated and oil is collected by gravity using a thin porous sponge sheet for wrapping the gel composition.

### EXAMPLE 21

Example 19 is repeated and oil is collected by gravity using a thin porous fabric for wrapping the gel composition.

### EXAMPLE 22

Example 19 is repeated and oil is collected by gravity using a thin sheet of expanded Teflon (Gore-tex) for wrapping the gel composition.

### EXAMPLE 23

Example 19 is repeated using a polyester fabric for wrapping the gel composition.

### EXAMPLE 24

Example 19 is repeated and oil is collected by gravity using a cheese cloth cotton fabric for wrapping the gel composition.

### EXAMPLE 25

Example 19 is repeated and oil is collected by gravity using a nylon fabric for wrapping the gel composition.

### EXAMPLE 26

Example 19 is repeated and oil is collected by gravity using thin 2 mm thick sheet of silicone elastomer made from GE LSR 29X0 for wrapping the gel composition.

### EXAMPLE 27

Example 19 is repeated and oil is collected by gravity using thin 2 mm thick sheet of silicone elastomer made from GE LSR3Z85/XX for wrapping the gel composition.

### EXAMPLE 28

Example 19 is repeated and oil is collected by gravity using thin 2 mm thick sheet of silicone elastomer made from GE LSR3Z86/XX for wrapping the gel composition.

### EXAMPLE 29

Example 19 is repeated and oil is collected by gravity using thin sheet of silicone elastomer in combination with a sheet of fabric for wrapping the gel composition.

### EXAMPLE 30

Examples 19-27 are repeated and oil is collected by gravity from a gel composition which gel composition is made from 2,000 parts by weight of oil per 100 parts by weight of polymer and using a sheet of fabric for wrapping the gel composition.

### EXAMPLE 31

Examples 30 are repeated and oil is collected by gravity from a gel composition which gel composition is made from 2,000 parts by weight of oil per 100 parts by weight of polymer and using a sheet of silicone elastomer reinforced fabric sheet coated on the inside with silicone fluid for wrapping the gel composition.

### EXAMPLE 32

Examples 19-28 are repeated and a refined oil mixture is collected by gravity from a medium crude oil mixture using a 200 cSt., silicone fluid for extraction of the crude oil mixture.

### EXAMPLE 33

Examples 19-28 are repeated and a refined oil mixture is collected by gravity from a medium crude oil mixture using a 200 cSt., silicone fluid for extraction of the raw crude oil mixture.

### EXAMPLE 34

An oil extended gel strand for flossing made from 400 parts by weight of Witco 40 oil and 100 parts by weight of Septon SEEPS 4055 is placed in a covered #1029 100×15 mm clear petri dish containing Dow corning dimethylpolysiloxane 200 fluid having a 100 cSt viscosity at room temperature for 30 hours, the resulting gel floss is found to exhibit a higher gel exterior rigidity, decrease in weight, and a greater gel tear strength as compared to the original floss.

### EXAMPLE 35

An oil extended gel strand for flossing made from 400 parts by weight of Duraprime 70 and 100 parts by weight of Septon SEEPS 4055 is placed in a covered #1029 100×15 mm clear petri dish containing Dow corning dimethylpolysiloxane 200 fluid having a 100 cSt viscosity at room temperature for 30 hours, the resulting gel floss is found to exhibit a higher gel exterior rigidity, decrease in weight, and a greater gel tear strength as compared to the original floss.

### EXAMPLE 36

An oil extended gel strand for flossing made from 400 parts by weight of Duraprime 55 and 100 parts by weight of Septon SEEPS 4055 is placed in a covered #1029 100×15 mm clear petri dish containing Dow corning dimethylpolysiloxane 200 fluid having a 100 cSt viscosity at room temperature for 30 hours, the resulting gel floss is found to exhibit a higher gel exterior rigidity, decrease in weight, and a greater gel tear strength as compared to the original floss.

### EXAMPLE 37

The following oil extended gel articles: a gel hand exercising grip, a gel shape floss suitable for use as a dental floss, a gel cushion, a gel pillow, a gel wrist rest, a gel leg rest, a gel neck cushion, a gel mattress, a gel bed pad, a gel elbow pad, a gel dermal pad, a gel wheelchair cushion, a gel helmet liner, a gel cold and hot pack, a gel exercise weight belt, a gel traction pad or belt, a gel cushion for splints, a gel sling, a gel brace for the hand, wrist, finger, forearm, knee, leg, clavicle, shoulder, foot, ankle, neck, back, rib, a gel sole for orthopedic shoe, a gel shaped toy article, a gel optical cladding for cushioning optical fibers from bending stresses, a gel swab tip, a gel fishing bate, a gel seal against pressure, a gel thread, a gel strip, a gel yarn, a gel tape, a weaved gel cloth, a gel fabrics, a gel balloon for valvuloplasty of the mitral valve, a gel trointestinal balloon dilator, a gel esophageal balloon dilator, a gel dilating balloon catheter use in coronary angiogram, a gel condom, a gel toy balloon, a gel surgical and examination glove, a self sealing enclosures for splicing electrical and telephone cables and wires, a gel film, and a gel liner made from 600 parts by weight of Witco 40 oil and 100 parts by weight of Septon SEEPS 4055 is placed in a bath containing Dow coming dimethylpolysiloxane 100 fluid having a 100 cSt viscosity at room temperature for 24 hours, the resulting gel floss is found to exhibit a higher gel exterior rigidity, decrease in weight, and a greater gel tear strength as compared to the original articles.

### EXAMPLE 38

A following thermoplastic oil gel composite articles are made with gel denoted by G, and physically interlocked with a selected material M in combination with one or more different gels forming the composite with the combination GnGn, GnGnGn, GnMn, GnMnGn, MnGnMn, MnGnGn, MnGnGnMn, GnMnGnGn, GnGnMnMn, GnMnMnGn, GnGnMnGnMnGnGn, GnMnGnMnMn, MnGnMnGnMnGn, GnGnMnMnGn, GnGnMnGnMn, GnGnMnGnMnGn, GnMnGnMnGn, and MnMnMnGn; where the M material is paper, foam, fabric, and synthetic fibers; and the gel is made from 400, 600, and 800 parts by weight respectively of Witco 40 oil and 100 parts by weight of Septon SEEPS 4055 is placed in a bath containing Dow corning dimethylpolysiloxane 200 fluid having a 100 cSt viscosity at room temperature for 30 hours, the resulting gel composites are found to exhibit a higher gel exterior rigidity, decrease in weight, and a greater gel tear strength as compared to the composite articles.

While certain features of this invention have been described in detail with respect to various embodiments thereof, it will, of course, be apparent that other modifications can be made within the spirit and scope of this invention, and it is not intended to limit the invention to the exact details shown above except insofar as they are defined in the following claims.

What I claim is:

1. A thermoplastic oil gel article comprising a gel with at least two different rigidity regions, said gel rigidity regions having no physically separable boundaries.

2. A thermoplastic oil gel article comprising a gel with at two or more rigidity regions, said gel rigidity regions having no physically separable boundaries.

3. A thermoplastic oil gel article comprising a gel with a first gel rigidity region continuous with a second gel rigidity region, said first gel rigidity region having a gel rigidity greater than said second gel rigidity region, said first and second gel rigidity regions separated by a continuous varying gel rigidity index region having physically inseparable boundaries.

4. A thermoplastic oil gel composite article comprising a gel with two or more rigidity regions, said gel rigidity regions having no physically separable boundaries, wherein said gel is being denoted by G, is physically interlocked with a selected material M or in combination with one or more of a different gel forming a composite of the combination GnGn, GnGnGn, GnMn, GnMnGn, MnGnMn, MnGnGn, MnMnMnGnMn, MnGnGnMn, GnMnGnGn, GnGnMnMn, GnMnMnGn, GnGnMnGnMnGnGn, GnMnGnMnMn, MnGnMnGnMnGn, GnGnMnMnGn, GnGnMnGnMn, GnGnMnGnMnGn, GnMnGnMnGn, MnMnMnGn or a permutation of one or more of said Gn with Mn; wherein when n is a subscript of M, n is the same or different selected from the group consisting of paper, foam, plastic, fabric, metal, metal foil, concrete, wood, glass, glass fibers, ceramics, synthetic resin, synthetic fibers or refractory materials; and wherein when n is a subscript of G, n denotes a different gel rigidity.

5. A thermoplastic oil gel composite article comprising a gel with two or more rigidity regions, said gel rigidity regions having no physically separable boundaries, wherein said gel is being denoted by G, is physically interlocked with a selected material M or in combination with one or more of a different gel forming a composite of the combination GnGn, GnGnGn, GnMn, GnMnGn, MnGnMn, MnGnGn, MnMnMnGnMn, MnGnGnMn, GnMnGnGn, GnGnMnMn, GnMnMnGn, GnGnMnGnMnGnGn, GnMnGnMnMn, MnGnMnGnMnGn, GnGnMnMnGn, GnGnMnGnMn, GnGnMnGnMnGn, GnMnGnMnGn, MnMnMnGn or a permutation of one or more of said Gn with Mn; wherein when n is a subscript of M, n is the same or different selected from the group consisting of paper, foam, fabric, or synthetic fibers; and wherein when n is a subscript of G, n denotes a different gel rigidity.

6. A gel article according to claim 1, wherein said gel article is a hand exercising grip, a gel shape floss suitable for use as a dental floss, a gel cushion, a gel pillow, a gel wrist rest, a gel leg rest, a gel neck cushion, a gel mattress, a gel bed pad, a gel elbow pad, a gel dermal pad, a gel wheelchair cushion, a gel helmet liner, a gel cold and hot pack, a gel exercise weight belt, a gel traction pad or belt, a gel cushion for splints, a gel sling, a gel brace for the hand, wrist, finger, forearm, knee, leg, clavicle, shoulder, foot, ankle, neck, back, rib, a gel sole for orthopedic shoe, a gel shaped toy article, a gel optical cladding for cushioning optical fibers from bending stresses, a gel swab tip, a gel fishing bate, a gel seal against pressure, a gel thread, a gel strip, a gel yarn, a gel tape, a weaved gel cloth, a gel fabrics, a gel balloon for valvuloplasty of the mitral valve, a gel trointestinal balloon dilator, a gel esophageal balloon dilator, a gel dilating balloon catheter use in coronary angiogram, a gel condom, a gel toy balloon, a gel surgical and examination glove, a self sealing enclosures for splicing electrical and telephone cables and wires, a gel film, or a gel liner.

8. A gel composite article according to claim 5, wherein said composite being formed into a gel hand exercising grip, a gel shape floss suitable for use as a dental floss, a gel crutch cushion, a gel cervical pillow, a gel bed wedge pillow, a gel leg rest, a gel neck cushion, a gel mattress, a gel bed pad, a gel elbow pad, a gel dermal pad, a gel wheelchair cushion, a gel helmet liner, a gel cold and hot pack, a gel exercise weight belt, a gel traction pad or belt, a gel cushion for splints, a gel sling, a gel brace for the hand, wrist, finger, forearm, knee, leg, clavicle, shoulder, foot, ankle, neck, back, rib, a gel sole for orthopedic shoe, a gel shaped toy article, a gel optical cladding for cushioning optical fibers from bending stresses, a gel swab tip, a gel fishing bate, a gel seal against pressure, a gel thread, a gel strip, a gel yarn, a gel tape, a weaved gel cloth, a gel fabrics, a gel balloon for valvuloplasty of the mitral valve, a gel trointestinal balloon dilator, a gel esophageal balloon dilator, a gel dilating balloon catheter use in coronary angiogram, a gel condom, a gel toy balloon, a gel surgical and examination glove, a self sealing enclosures for splicing electrical and telephone cables and wires, a gel film, or a gel liner.

9. A process of extracting oil from a oil containing material comprising the steps of:
(1) adding to a container an amount of silicone fluid having a selected viscosity to a predetermined measured silicone fluid level for receiving said material,
(2) submerging said material in said silicone fluid for a selected time,
(3) collecting said extracted oil from said container above said silicone fluid level for a selected period of time.

10. A process of extracting oil from a oil containing solid material body comprising the steps of:
(1) coating an amount of silicone fluid of a selected viscosity over the surface of said material body,
(2) suspending said material body in a vertical position over a drain for collecting said extracting oil for a selected period of time.

11. A process of extracting oil from a oil containing solid material body comprising the steps of:
(1) saturating a durable sheet of porous material with a selected amount of silicone fluid having a selected viscosity to form a silicone sheet,
(1) wrapping said material body with said silicone sheet, and
(2) suspending said silicone sheet wrapped material body in a vertical position over a drain for collecting said extracting oil for a selected period of time.

12. A process of extracting oil from a oil containing solid material body comprising the steps of:
(1) saturating a durable sheet of porous material with a selected amount of silicone fluid having a selected viscosity to form a silicone sheet,
(1) wrapping said material body with said silicone sheet, and
(2) suspending said silicone sheet wrapped material body on a flat incline surface over a drain for collecting said extracting oil from said incline surface for a selected period of time.

13. A process of extracting oil from a oil containing solid material body comprising the steps of:
(1) coating an amount of silicone fluid of a selected viscosity over the surface of said material body,
(2) supporting said material body on a flat incline surface over a drain for collecting said extracting oil from said incline surface for a selected period of time.

14. A process of extracting oil from a oil containing solid material body comprising the steps of:
(1) coating a selected amount of silicone fluid having a selected viscosity on a flat surface of said material body,
(2) supporting said silicone fluid coated flat surface of said material body on a flat incline surface over a drain for collecting said extracting oil from said incline surface for a selected period of time.

15. A process of extracting oil from a oil containing material body comprising the steps of:
(1) adding to a container an amount of silicone fluid having a selected viscosity to a predetermined measured silicone fluid level for receiving said material body,
(2) coating said material with said silicone fluid by turning said material body over in said container of said silicone fluid and allow said material body to rest in said container for a selected time, and
(3) collecting said extracted oil from said container above said silicone fluid level for a selected period of time.

16. A process of extracting oil from a oil containing material comprising the steps of:
(1) saturating a durable sheet of porous material with a selected amount of silicone fluid having a selected viscosity to form a silicone sheet,
(2) wrapping said material with said silicone sheet,
(3) adding to a container an amount of silicone fluid having a selected viscosity to a predetermined measured silicone fluid level for receiving said material,
(4) submerging said silicone sheet wrapped material in said silicone fluid,
(5) collecting said extracted oil from said container above said silicone fluid level for a selected period of time.

17. A process according to claim 16, wherein said material is a corn, grain, a seed, a plant, a bean, or a biomass.

18. A process according to claim 16, wherein said material is a heavy crude oil.

19. A process according to claim 15, wherein said material is an oil extended polymer gel.

20. A process according to claim 12, wherein said material is an oil extended polymer gel.
